(19) United States
(12) Patent Application Publication
(10) Pub. No.: US 2023/0126099 A1
(43) Pub. Date: Apr. 27, 2023

(54) ENDOSCOPE SYSTEM, CONTROLLER, AND STAND

(71) Applicant: OLYMPUS CORPORATION, Tokyo (JP)

(72) Inventor: Yasuo FUNAKOSHI, Hachioji-shi (JP)

(73) Assignee: OLYMPUS CORPORATION, Tokyo (JP)

(21) Appl. No.: 17/969,098

(22) Filed: Oct. 19, 2022

Related U.S. Application Data
(60) Provisional application No. 63/272,338, filed on Oct. 27, 2021.

(51) Int. Cl. A61B 1/00; A61B 50/13; A61B 1/005; A61B 1/018

(52) U.S. Cl. CPC A61B 1/0016; A61B 50/13; A61B 1/00114; A61B 1/0052; A61B 1/018; A61B 1/015

(57) ABSTRACT

An endoscope system includes: a drive cable; an endoscope that has a bending portion that is capable of bending and is driven by the connected drive cable; a drive device that electrically drives the connected drive cable; a trolley equipped with the drive device; a stand that connects the drive cable and the endoscope and is capable of being installed at a position away from the trolley; and an operation device that is communicably connected to the drive device and attached to the stand.

The present application claims priority based on U.S. Patent Provisional Application No. 63/272,338 provisionally filed in the United States on Oct. 27, 2021, the contents of which are incorporated herein by reference.

### BACKGROUND

### Technical Field

The present disclosure relates to an endoscope system.

### Background Art

Conventionally, an endoscope has been used for observation and treatment in luminal organs such as the digestive tract. There is a demand for an endoscope system that can perform observation and treatment using the endoscope more efficiently. In the manipulator system described in Japanese Patent No. 5192898, since a manipulator provided with the endoscope is electrically driven, observation and treatment by the endoscope can be efficiently performed.

In an endoscope system equipped with an electrically driven endoscope, it is desired that the operation portion (controller), to which operation by a surgeon is input, is easy to handle and can be operated intuitively.

### SUMMARY

According to a first aspect of the present invention, an endoscope system includes: a drive cable; an endoscope that has a bending portion that is capable of bending and is driven by the connected drive cable; a drive device that electrically drives the connected drive cable; a trolley equipped with the drive device; a stand that connects the drive cable and the endoscope and is capable of being installed at a position away from the trolley; and an operation device that is communicably connected to the drive device and attached to the stand.

According to a second aspect of the present invention, a controller controls an endoscope including a bending portion that is capable of bending, and includes a support portion; a grip: a shaft provided in front of the grip; and an operation input portion which is supported by the support portion and to which an operation of bending the bending portion by rotating the shaft along a longitudinal axis is input.

### BRIEF DESCRIPTION OF THE DRAWINGS

FIG. 1 is an overall view of an endoscope system according to a first embodiment.

FIG. 2 is a diagram showing an endoscope and an operation device of the endoscope system used by a surgeon.

FIG. 3 is a diagram showing an insertion portion of the endoscope.

FIG. 4 is a cross-sectional view of the bending portion.

FIG. 5 is a diagram showing the operation device.

FIG. 6 is a diagram showing the operation device.

FIG. 7 is a diagram showing the operation device.

FIG. 8 is a diagram showing the operation device held by a left hand.

FIG. 9 is a diagram showing the operation device in which an LR operation input portion is operated.

FIG. 10 is a functional block diagram of a drive device of the endoscope system.

FIG. 11 is a functional block diagram of a video control device of the endoscope system.

FIG. 12 is a diagram showing the operation device and a treatment tool that are operated at the same time.

### DETAILED DESCRIPTION OF THE PREFERRED EMBODIMENTS

### First Embodiment

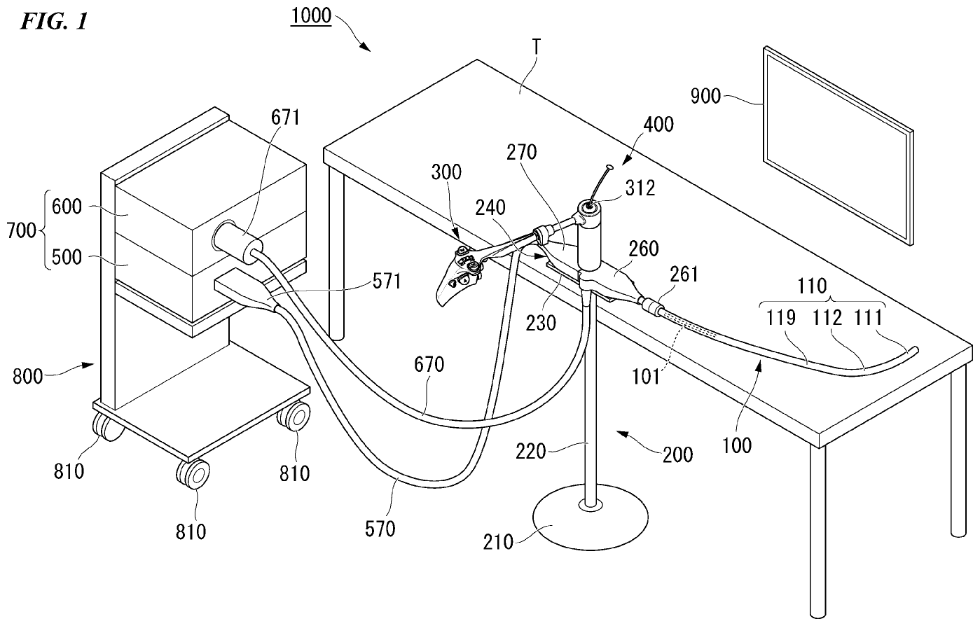

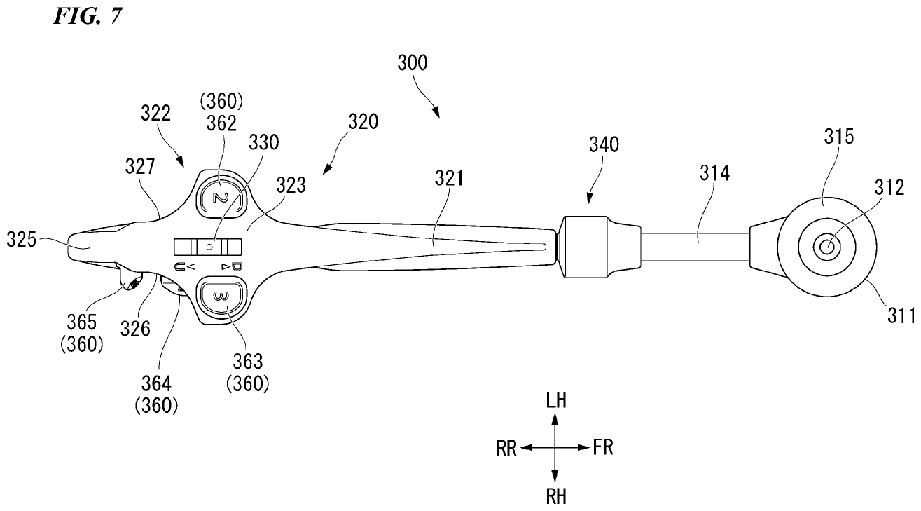

An endoscope system 1000 according to a first embodiment of the present invention will be described with reference to FIGS. 1 to 12. FIG. 1 is an overall view of the endoscope system 1000 according to the present embodiment.

### [Endoscope System 1000]

The endoscope system 1000 is a medical system used to observe and treat the inside of a patient P lying on an operation table T. The endoscope system 1000 includes an endoscope 100, a stand 200, an operation device 300, a treatment tool 400, a drive device 500, a video control device 600, a trolley 800, and a display device 900. A drive cable 570 is connected to the drive device 500. A video control cable 670 is connected to the video control device 600.

The endoscope 100 is a device that is inserted into the lumen of the patient P to observe and treat the affected area. The endoscope 100 is connected to the drive device 500 and the video control device 600 via a connector 240 included in the stand 200. An internal path 101 is formed inside the endoscope 100. In the following description, in the endoscope 100, the side inserted into the lumen of the patient P is referred to as “distal end side A1”, and the side connected to the connector 240 is referred to as “proximal end side A2”.

The stand 200 can be arranged at a position away from the trolley 800, and has the connector 240 for connecting the endoscope 100 to the drive cable 570 and the video control cable 670.

The operation device 300 is a device for inputting the operation of the surgeon who controls the endoscope system 1000. The operation device 300 is fixed to the upper part of the stand 200. The operation device 300 is connected to the drive device 500 via an operation cable (not shown) arranged in the drive cable 570. The operation device 300 may be able to communicate with the drive device 500 by wireless communication instead of wired communication. The surgeon S can electrically drive the endoscope 100 by operating the operation device 300.

The treatment tool 400 is a device that inserts the internal path 101 of the endoscope 100 into the lumen of the patient P to treat the affected area. In FIG. 1, the treatment tool 400 is inserted into the internal path 101 of the endoscope 100 via a forceps opening 312 formed in the operation device 300.

The drive device 500 is detachably connected to the drive cable 570. The drive device 500 is connected to the endoscope 100 via the drive cable 570 and the connector 240. The drive device 500 drives the endoscope 100 by electrically driving the drive cable 570 using a built-in motor based on the operation input to the operation device 300. Further, the drive device 500 drives a built-in pump or the like based on the operation input to the operation device 300 to cause the endoscope 100 to perform air supply suction.

The video control device 600 is detachably connected to the video control cable 670. The video control device 600 is connected to the endoscope 100 via the video control cable 670 and the connector 240, and acquires a captured image from the endoscope 100. The video control device 600 causes the display device 900 to display the captured image acquired from the endoscope 100 and the GUI image or the CG image for the purpose of providing information to the surgeon.

The drive device 500 and the video control device 600 constitute a control device 700 that controls the endoscope system 1000. The control device 700 may further include peripheral devices such as a video printer. The drive device 500 and the video control device 600 may be an integrated device.

The trolley 800 is a trolley having wheels 810, and is equipped with the control device 700 (drive device 500 and video control device 600). The trolley 800 may be equipped with, for example, only the drive device 500. Further, the trolley 800 may have a hanger on which the operation device 300 and the like can be suspended.

The display device 900 is a device capable of displaying an image such as an LCD. The display device 900 is connected to the video control device 600 via a display cable.

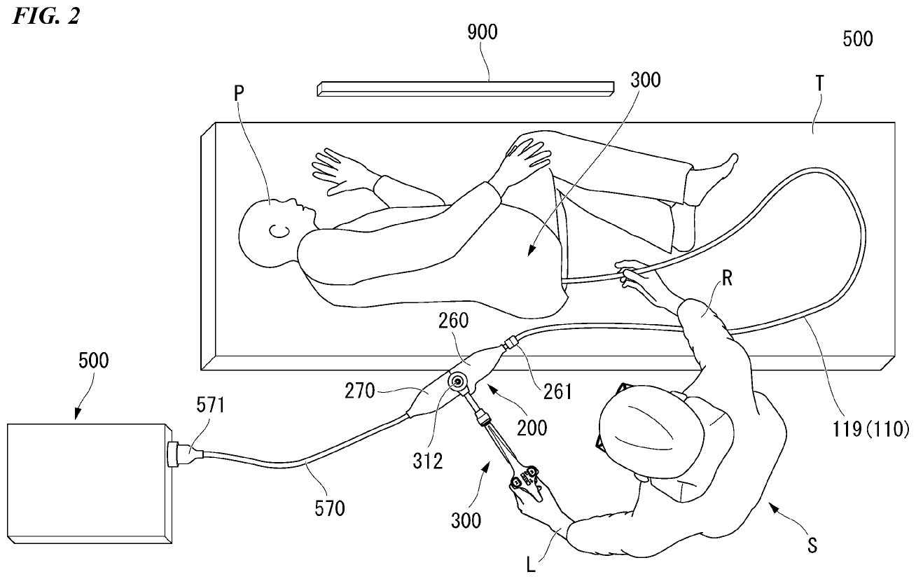

FIG. 2 is a diagram showing the endoscope 100 and the operation device 300 used by the surgeon S.

For example, the surgeon S operates the operation device 300 with the left hand L, for example, while observing the captured image displayed on the display device 900, while operating the endoscope 100 inserted into the lumen from the anus of the patient P. Since the endoscope 100 and the operation device 300 are separated, the surgeon S can operate the endoscope 100 and the operation device 300 independently without being affected by each other.

Next, each device of the endoscope system 1000 will be described in detail.

### [Endoscope 100]

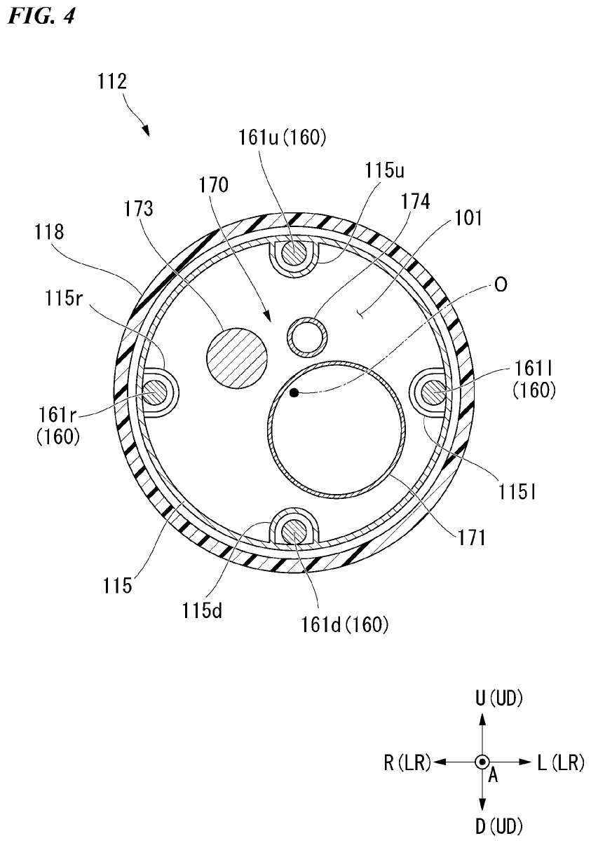

As shown in FIG. 1, the endoscope 100 includes an insertion portion 110, a curved wire 160 (see FIG. 4), and an internal component 170 (see FIG. 4). The proximal end of the endoscope 100 is connected to the connector 240.

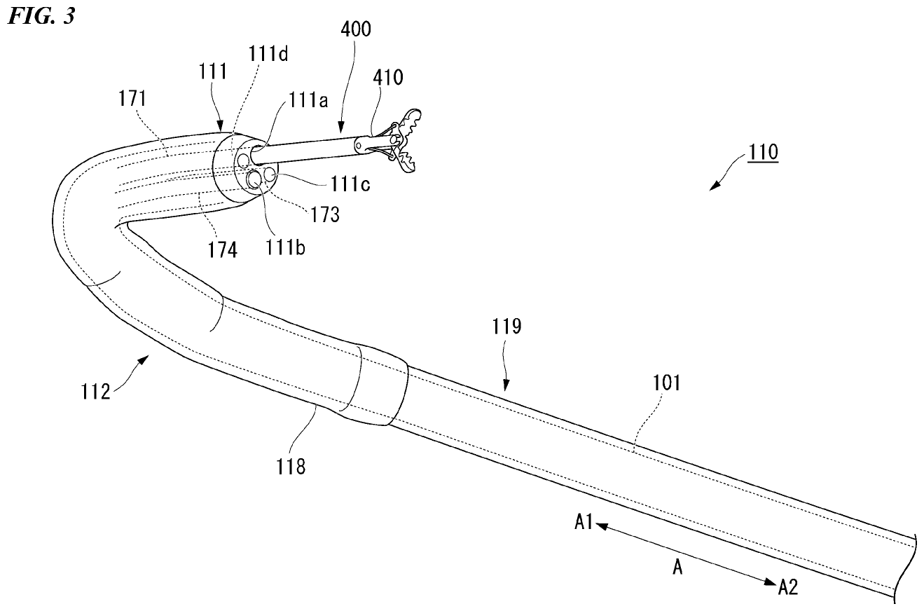

FIG. 3 is a diagram showing the insertion portion 110 of the endoscope 100.

Inside the endoscope 100, the internal path 101 extending from the distal end end of the insertion portion 110 to the proximal end along the longitudinal direction A of the endoscope 100 is formed. The curved wire 160 and the internal structure 170 are inserted into the internal path 101. The built-in object 170 includes a channel tube 171, an imaging cable 173, and a light guide 174.

The insertion portion 110 is an elongated long member that can be inserted into the lumen. The insertion portion 110 has a distal end portion 111, a bending portion 112, and an internal soft portion 119. The distal end portion 111, the bending portion 112, and the internal soft portion 119 are connected in order from the distal end side.

The distal end portion 111 is formed in a substantially cylindrical shape by metal or the like. As shown in FIG. 3, the distal end portion 111 has an opening portion 111a, an illumination portion 111b, and an imaging portion 111c. The opening 111a is an opening that communicates with the channel tube 171. As shown in FIG. 3, a treatment portion 410 such as a gripping forceps provided at the distal end of the treatment tool 400 through which the channel tube 171 is inserted is recessed from the opening 111a.

The illumination portion 111b is connected to the light guide 174 that guides the illumination light, and emits illumination light that illuminates the imaging target. The imaging portion 111c includes an imaging element such as CMOS, and takes an image of an imaging target. The imaging signal is sent to the video control device 600 via the imaging cable 173 and the video control cable 670.

FIG. 4 is a cross-sectional view of the bending portion 112.

The bending portion 112 has a plurality of knot rings (also referred to as curved pieces) 115. The plurality of knot rings 115 are connected so as to be bendable in the longitudinal direction A. The bending portion 112 can be curved in a vertical direction (also referred to as “UD direction”) perpendicular to the longitudinal direction A and in a left-right direction (also referred to as “LR direction”) perpendicular to the longitudinal direction A and the UD direction.

An upper wire guide 115u, a lower wire guide 115d, a left wire guide 115l, and a right wire guide 115r are formed on the inner peripheral surface of the knot ring 115. The upper wire guide 115u and the lower wire guide 115d are arranged on both sides in the UD direction with the central axis O in the longitudinal direction A interposed therebetween. The left wire guide 115l and the right wire guide 115r are arranged on both sides in the LR direction with the central axis O in the longitudinal direction A interposed therebetween.

The curved wire 160 is a wire that bends the bending portion 112. The curved wire 160 extends through the internal path 101 to the connector 240. The curved wire 160 has an upper curved wire 161u, a lower curved wire 161d, a left curved wire 161l, and a right curved wire 161r.

The upper curved wire 161u and the lower curved wire 161d are wires that bend the bending portion 112 in the UD direction. The upper curved wire 161u is inserted with the upper wire guide 115u. The lower curved wire 161d is inserted with the lower wire guide 115d. The distal ends of the upper curved wire 161u and the lower curved wire 161d are fixed to the distal end portion 111.

The left curved wire 161l and the right curved wire 161r are wires that bend the bending portion 112 in the LR direction. The left curved wire 161l is inserted with the left wire guide 115l. The right curved wire 161r is inserted with the right wire guide 115r. The distal ends of the left curved wire 161l and the right curved wire 161r are fixed to the distal end portion 111.

The bending portion 112 can be bent in a desired direction by pulling or relaxing the curved wire 160 (upper curved wire 161u, lower curved wire 161d, left curved wire 161l, right curved wire 161r).

The internal soft portion 119 is along and flexible tubular member. The curved wire 160, the channel tube 171 and the imaging cable 173 and the light guide 174 are inserted into the internal path 101 formed in the internal soft portion 119.

### [Stand 200]

The stand 200 is a self-standing stand that can be placed at a position away from the trolley 800. The stand 200 includes a pedestal 210, a pole 220, a support plate 230, and a connector 240.

The pedestal 210 is a leg for supporting the pole 220. The pedestal 210 illustrated in FIG. 1 is formed in a disk shape, but the pedestal 210 may have another shape as long as it can support the pole 220. The pole 220 is a support column that stands up from the pedestal 210 and supports the support plate 230. The pole 220 supports the support plate 230 and the connector 240 at a position higher than the operation table T.

The support plate 230 is provided on the upper part of the pole 220 and supports the connector 240. The support plate 230 is rotatably attached to the pole 220 in the horizontal direction. The connector 240 supported by the support plate 230 is rotatable in the horizontal direction.

The connector 240 detachably connects the drive cable 570 and the endoscope 100. Further, the video control cable 670 and the endoscope 100 are detachably connected to each other. The connector 240 has a first connector 260, and a second connector 270.

The first connector 260 is a connector to which the proximal end of the internal soft portion 119 of the endoscope 100 is attached. The first connector 260 has a rotary connection connector 261. The rotary connection connector 261 connects the internal soft portion 119 to the first connector 260 so as to be rotatable with respect to the longitudinal axis of the internal soft portion 119. The first connector 260 is detachable from the second connector 270. Further, the first connector 260 is detachable from the distal end of the video control cable 670.

The second connector 270 is a connector to which the distal end of the drive cable 570 is attached. The second connector 270 is detachable from the first connector 260.

The mounted first connector 260 and second connector 270 can be installed on the support plate 230 as shown in FIG. 1. The first connector 260 and the second connector 270 are fixed so as not to be detachable by installing them on the support plate 230. The first connector 260 and the second connector 270 can be attached and detached by removing them from the support plate 230.

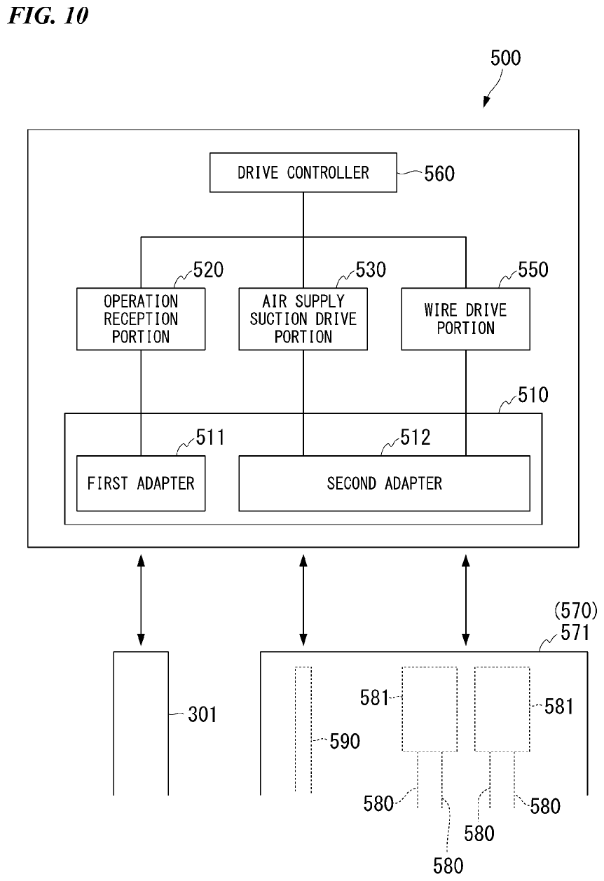

When the first connector 260 and the second connector 270 are attached, a drive wire 580 (see FIG. 10) of the drive cable 570 is connected so that the curved wire 160 of the endoscope 100 can be pulled and relaxed. Further, an air supply suction tube 590 (see FIG. 10) of the drive cable 570 is connected to the channel tube 171 of the endoscope 100.

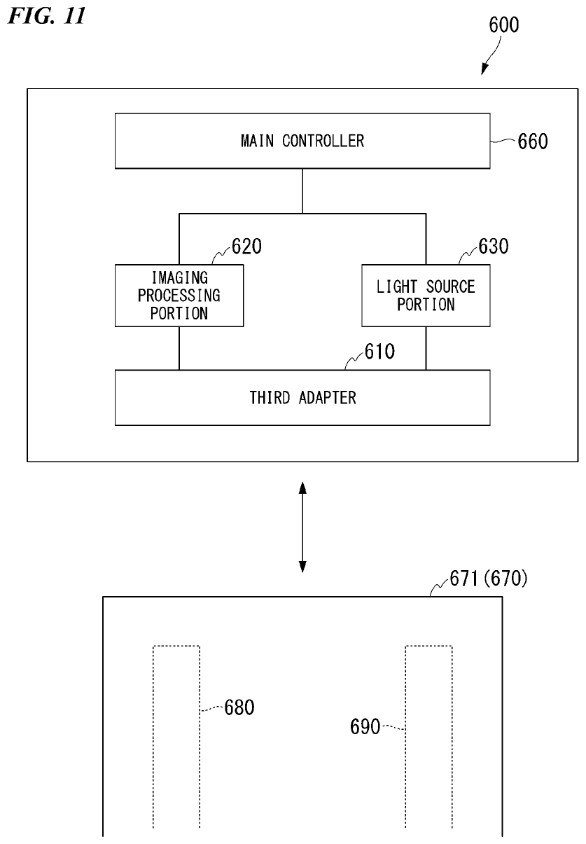

When the video control cable 670 is attached to the first connector 260, a video cable 680 (see FIG. 11) of the video control cable 670 is connected to the imaging cable 173 of the endoscope 100. Further, a light cable 690 (see FIG. 11) of the video control cable 670 is connected to the light guide 174 of the endoscope 100.

### [Operation Device 300]

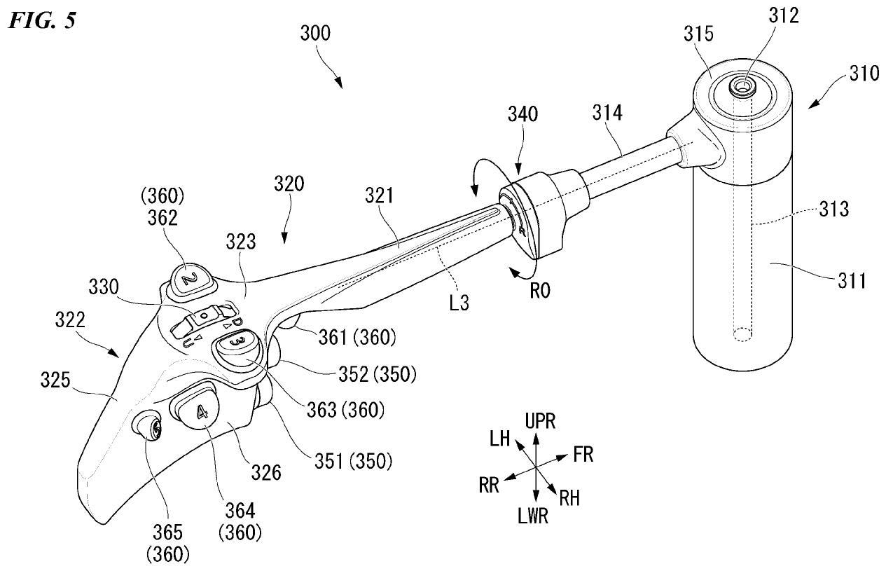

FIGS. 5 to 7 are views showing the operation device 300.

The operation device (controller) 300 is a device to which an operation (particularly, an operation of driving the endoscope 100) of an operator who controls the endoscope system 1000 is input. The input operation input is transmitted to the drive device 500 via an operation cable (not shown).

The operation device 300 includes a support portion 310, an operation portion main body 320, a UD operation lever 330, an LR operation input portion 340, a main operation button 350, and a sub operation button 360.

In the following description, the longitudinal direction along the longitudinal axis L3 of the operation device 300 is defined as the “front-back direction”, and the direction in which the grip that grips the operation portion main body 320 is attached is defined as the “rear RR”, whose opposite direction is defined as “forward FR”. The direction in which the operation device 300 is attached to the stand 200 is defined as “upward UPR”, whose opposite direction is defined as “downward LWR”. The direction toward the upper UPR or the lower LWR is defined as “vertical direction”. The rightward direction toward the rear RR is defined as “right RH”, whose opposite direction is defined as “left LH”. The direction toward the right RH or the left LH is defined as “left-right direction”.

The support portion 310 is connected to the stand 200 and supports the operation portion main body 320. The support portion 310 may be fixed to the stand 200 or may be detachably connected to the stand 200. The support portion 310 has a support column 311 and an arm 314.

The support column 311 is a support column formed in a columnar shape extending in the vertical direction. The support column 311 has the forceps opening 312. The forceps opening 312 is an opening provided in an upper surface 315, which is the end face of the upper UPR. The forceps opening 312 communicates with the channel tube 171 of the endoscope 100 through which the stand 200 is inserted via an insertion passage 313 formed in the support column 311. The support column 311 and the pole 220 may be integrally formed.

The arm 314 is an arm that extends from the support portion 310 in a direction perpendicular to the direction in which the support column 311 extends and along the longitudinal axis L3. The arm 314 is rotatably attached to the support column 311 with the longitudinal axis of the support column 311 as a rotation axis. The arm 314 connects the support column 311 and the operation portion main body 320. By forming the arm 314 with a material (material having a lower rigidity) that is more flexible than the support column 311 and the operation portion main body 320, it is possible to allow the operation portion main body 320 to swing in the vertical direction with respect to the support column 311. As a result, even if the position of the left hand L of the surgeon S changes, the operation by the left hand L can be continued, so that the comfort of the operation is maintained. Further, when the arm 314 is made of a flexible material, there is an effect of preventing the connecting portion between the arm 314 and the support column 311 from being damaged by an external force. The support portion 310 does not have to have the arm 314. In that case, the operation portion main body 320 is directly connected to the support column 311 via the LR operation input portion 340.

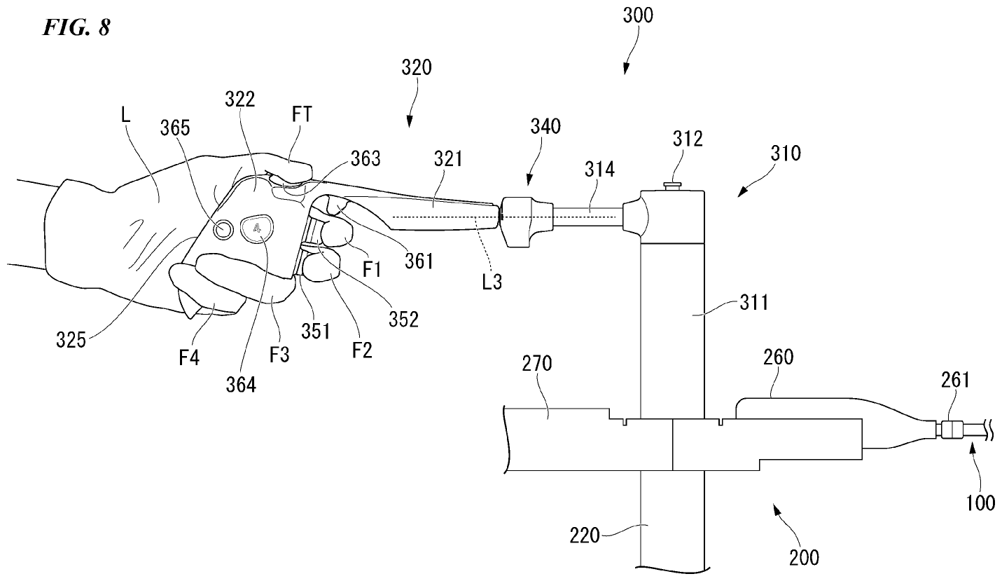

FIG. 8 is a diagram showing the operation device 300 held by the left hand L.

The operation portion main body 320 is formed in the shape of a hand gun and has a rotating shaft 321 and a grip 322. The operation portion main body 320 is held by the left hand L of the surgeon S. The operation portion main body 320 is supported by the support portion 310, and remains in the current position even when the surgeon S releases the left hand L from the operation portion main body 320.

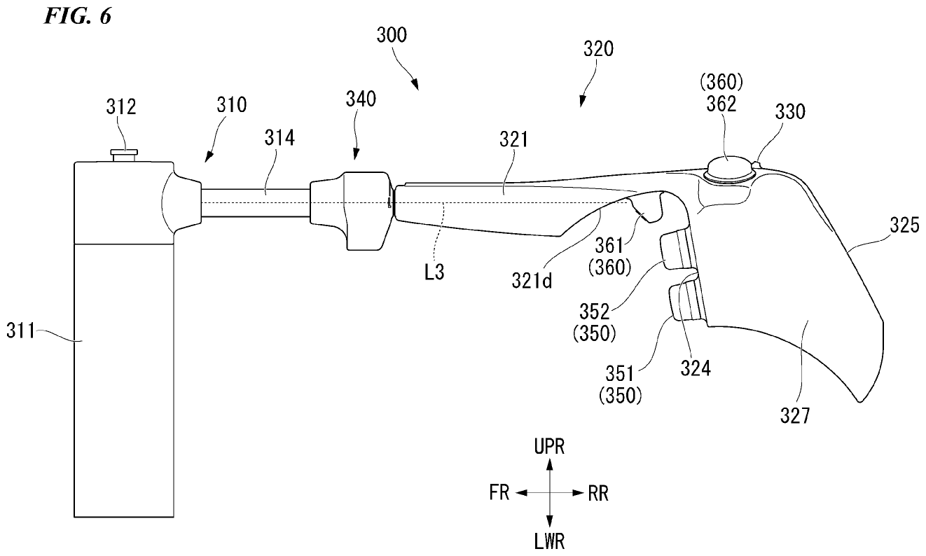

The rotary shaft 321 is formed in a substantially columnar shape extending along the longitudinal axis L3, and is provided in the front FR of the grip 322. As shown in FIG. 6, the rotary shaft 321 has a notch 321d at a connecting portion connected to the grip 322 in the rear RR. The notch portion 321d is a recess in which the lower LWR of the rotary shaft 321 is recessed. The rotary shaft 321 is connected to the LR operation input portion 340 in the front FR.

The grip 322 is formed in a gun grip shape that is easy to grip with one hand, and is provided on the rear RR of the rotating shaft 321. The grip 322 has an upper surface 323, a front surface 324, a back surface 325, a right side surface 326, and a left side surface 327. The upper surface 323 is a surface facing the upper UPR and is a surface continuous with the rotating shaft 321. The front surface 324 is a surface facing the front FR. The back surface 325 is an inclined surface extending from the upper surface 323 to the rear RR, and is a surface facing upward and rearward. The right side surface 326 is a surface facing the right RH. The left side surface 327 is a surface facing the left LH.

The UD operation lever 330 is provided on the upper surface 323 of the grip 322, and is a rotary lever to which an operation of bending the bending portion 112 in the UD direction is input. The UD operation lever 330 is provided so as to be rotatable in the front-rear direction. When the UD operation lever 330 is pushed into the front FR, the operation device 300 transmits an operation input for bending the bending portion 112 toward the lower side D to the drive device 500. When the UD operation lever 330 is pushed into the rear RR, the operation device 300 transmits an operation input for bending the bending portion 112 toward the upper U to the drive device 500. The direction (lower D/upper U) in which the bending portion 112 is curved may be reversed, which corresponds to the direction (front FR/rear RR) in which the UD operation lever 330 is pushed.

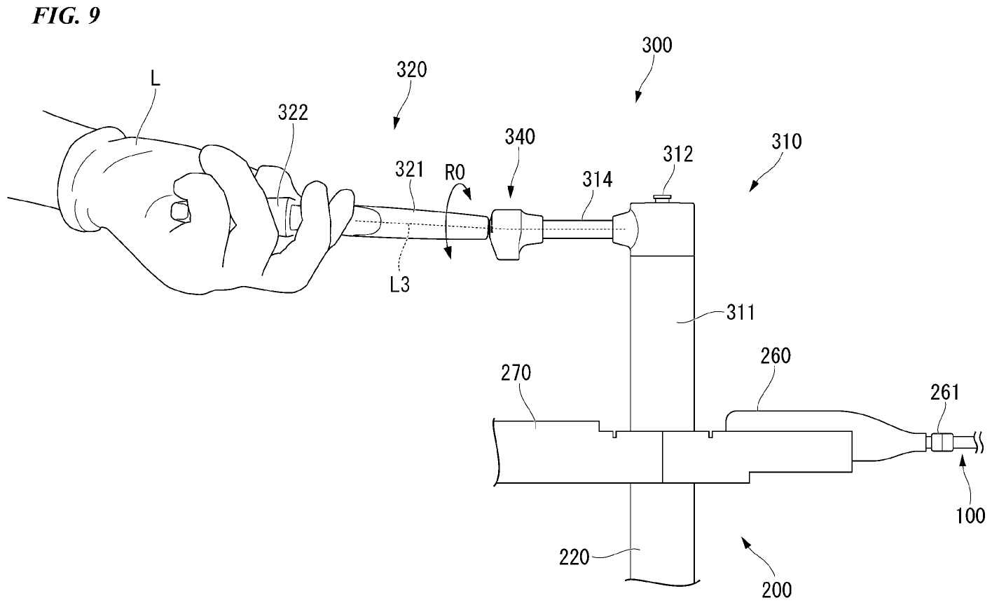

FIG. 9 is a diagram showing the operation device 300 in which the LR operation input portion 340 is operated.

The LR operation input portion 340 is provided on the rear RR of the arm 314, and is an operation input portion for inputting an operation of bending the bending portion 112 in the LR direction. The rotary shaft 321 is connected to the LR operation input portion 340 so that the longitudinal axis of the rotary shaft 321 substantially coincides with the longitudinal axis of the arm 314. The rotary shaft 321 is rotatably connected to the LR operation input portion 340 about the longitudinal axis L3. That is, the LR operation input portion 340 is a member that rotatably connects the rotary shaft 321 to the arm 314 about the longitudinal axis L3. More specifically, the LR operation input portion 340 is a bearing, a dial, a pulley, or the like, or a combination thereof. Further, the LR operation input portion 340 is not limited to these, and other known members may be used. The LR operation input portion 340 has an encoder or the like that can detect an angle (roll angle) at which the rotary shaft 321 rotates (roll rotation RO) about the longitudinal axis L3 with respect to the LR operation input portion 340.

As shown in FIG. 8, when the rotary shaft 321 is rotated to the left LH about the longitudinal axis L3 with respect to the LR operation input portion 340, the operation device 300 transmits to the drive device 500 an operation input for bending the bending portion 112 toward the left side L. When the rotary shaft 321 is rotated to the right RH with respect to the longitudinal axis L3 with respect to the LR operation input portion 340, the operation device 300 transmits to the drive device 500 an operation input for bending the bending portion 112 toward the right side R.

The main operation button 350 is a button for inputting an operation for the main function of the endoscope 100. The main operation button 350 has an air supply/water supply button 351 and a suction button 352. As shown in FIG. 6, the main operation button 350 is attached to the front surface 324 of the grip 322. The air supply/water supply button 351 and the suction button 352 are arranged side by side from the lower LWR toward the upper UPR.

The air supply/water supply button 351 is attached to the front surface 324 of the grip 322, and is a push button for inputting an operation of supplying air/water from the opening 111a of the distal end portion 111 of the endoscope 100. When the air supply/water supply button 351 is pushed in, the operation device 300 transmits an operation input for executing the air supply/water supply to the drive device 500.

The suction button 352 is attached to the front surface 324 of the grip 322, and is a push button to which an operation of performing suction is input from the opening 111a of the distal end portion 111 of the endoscope 100. When the suction button 352 is pushed in, the operation device 300 transmits an operation input for performing suction to the drive device 500.

The sub operation button 360 includes a first programmable button 361, a second programmable button 362, a third programmable button 363, a fourth programmable button 364, and a fifth programmable button 365. The sub operation button 360 is a push button into which an arbitrarily set operation is input.

As shown in FIG. 6, the first programmable button 361 is provided in the notch portion 321d of the rotary shaft 321. The first programmable button 361 is a button to which an operation is input by moving it to the rear RR like a trigger operation of a hand gun.

The second programmable button 362 and the third programmable button 363 are attached to the upper surface 323. The second programmable button 362 and the third programmable button 363 are provided on both sides in the left-right direction with the UD operation lever 330 interposed therebetween. The second programmable button 362 and the third programmable button 363 having surfaces to which the fingers touch are oriented in different directions. Specifically, the second programmable button 362 is arranged on the left LH (left side) with respect to the UD operation lever 330. The surface of the second programmable button 362 that the finger touches faces the direction (upper right direction) toward the UD operation lever 330 from the upper UPR. Further, the third programmable button 363 is arranged on the right RH (right side) with respect to the UD operation lever 330. The surface of the third programmable button 363 with which the finger touches faces the direction (upper left direction) toward the UD operation lever 330 side from the upper UPR. Therefore, the surgeon S can distinguish between the second programmable button 362 and the third programmable button 363 by tactile sensation without visually confirming.

The fourth programmable button 364 and the fifth programmable button 365 are attached to the right side surface 326. The fourth programmable button 364 and the fifth programmable button 365 have their finger contact surfaces facing different directions. Specifically, the surface of the fourth programmable button 364 that the finger touches faces upward UPR On the other hand, the surface of the fifth programmable button 365 that the finger touches faces the front FR. Further, the fourth programmable button 364 is larger than the fifth programmable button 365. Therefore, the surgeon S can distinguish between the fourth programmable button 364 and the fifth programmable button 365 by tactile sensation without visually confirming.

The fourth programmable button 364 and the fifth programmable button 365 are arranged on the side surface that can be operated with the thumb FT when the grip 322 is gripped. More specifically, the operation device 300 shown in FIGS. 5 and 6 has a configuration assuming an operation with the left hand L. In this case, when the grip 322 is gripped by the left hand L, the thumb FT of the gripped hand is located on the right side surface 326. Therefore, the fourth programmable button 364 and the fifth programmable button 365 are arranged on the right side surface 326 so that they can be operated by the thumb FT of the grasping hand. However, the arrangement of the fourth programmable button 364 and the fifth programmable button 365 is not limited to this. For example, assuming that the grip 322 is gripped by the right hand, the fourth programmable button 364 and the fifth programmable button 365 may be arranged on the left side surface 327.

As shown in FIG. 8, the surgeon S brings the palm of the left hand L into contact with the back surface 325, operates the UD operation lever 330 with the thumb FT, and operates the main operation button 350 and the first programmable button 361 with the index finger F1 and the middle finger F2.

The surgeon S can input the operation of the endoscope 100 as if operating a hand gun. Specifically, the surgeon S can operate the index finger F1 and the middle finger F2 with the main operation button 350 and the first programmable button 361 while holding the grip 322 with the left hand L, as in the trigger operation of a hand gun. Further, the surgeon S can operate the UD operation lever 330 with the thumb FT while holding the grip 322 with the left hand L, like the hammer operation of a hand gun.

As shown in FIG. 9, the surgeon S can input an operation of bending the bending portion 112 in the LR direction by rotating the operation portion main body 320 with respect to the LR operation input portion 340 by rolling around the longitudinal axis L3. Therefore, it is not necessary to arrange the button for inputting the operation of bending the bending portion 112 in the LR direction on the operation portion main body 320. Further, since the operation of rolling the operation portion main body 320 with respect to the LR operation input portion 340 is similar to the operation of bending the bending portion 112 in the LR direction, the surgeon S can intuitively input the operation of bending the bending portion 112 in the LR direction.

The surgeon S can operate the endoscope 100 in the same or similar operation mode as the operation mode for operating a conventional manual endoscope (operation input method for each operation input portion and assignment of the finger to be operated). Specifically, the surgeon S operates the UD operation lever 330 with the thumb FT and rolls the operation portion main body 320 with respect to the LR operation input portion 340 to operate the bending portion 112 of the endoscope 100. Even when operating a conventional manual endoscope, the surgeon S rotates the knob with the thumb FT of the left hand and rolls the manual endoscope operation portion with respect to the insertion tube of the endoscope. In many cases, the manual endoscope is curved by the operation. Further, the surgeon S operates the main operation button 350 with the index finger F1 and the middle finger F2. Even when operating a conventional manual endoscope, the surgeon S often operates the operation buttons in which various functions of the manual endoscope are set by the index finger F1 and the middle finger F2 of the left hand. Further, the surgeon S operates the air supply/water supply button 351 and the suction button 352 by the index finger F1 and the middle finger F2. Even when operating a conventional manual endoscope, the surgeon S often operates air supply/water supply and suction with the index finger F1 and the middle finger F2 of the left hand.

Further, regarding the main operation button 350 and the sub operation button 360, it is also possible to design the assignment and arrangement of functions so that the operation mode is common to or similar to the operation mode for operating a conventional manual endoscope. This makes it possible to provide an operation device having good operability even for an operator who is accustomed to the operation of a conventional manual endoscope.

### [Drive 500]

FIG. 10 is a functional block diagram of the drive device 500.

The drive device 500 includes an adapter 510, an operation reception portion 520, an air supply suction drive portion 530, a wire drive portion 550, and a drive controller 560.

The adapter 510 has a first adapter 511 and a second adapter 512. The first adapter 511 is an adapter to which an operation cable 301 is detachably connected. The second adapter 512 is an adapter to which a connecting portion 571 provided on the proximal end side of the drive cable 570 is detachably connected. The drive cable 570 includes a drive wire 580 and an air supply suction tube 590.

The operation reception portion 520 to which an operation is input from the operation device 300 via the operation cable. In a case where the operation device 300 and the driving device 50) communicate by wireless communication instead of wired communication, the operation reception portion 520 has a known wireless-receiving module.

The air supply suction drive portion 530 is connected to the air supply suction tube 590 of the drive cable 570. The air supply suction drive portion 530 includes a pump or the like, and supplies air or liquid to the air supply suction tube 590. Further, the air supply suction drive portion 530 sucks air from the air supply suction tube 590.

The wire drive portion 550 is connected to the drive wire 580 of the drive cable 570 via a drive wire adapter 581. The wire drive portion 550 has a drive portion and an encoder (not shown). The drive portion pulls or relaxes the drive wire 580 of the drive cable 570 by a pulley or the like. The encoder detects the traction amount of the drive wire 580. The detection result of the encoder is acquired by the drive controller 560 of the drive device 500.

The drive controller 560 controls the entire drive device 500. The drive controller 560 acquires the operation input received by the operation reception portion 520. The drive controller 560 controls the air supply suction drive portion 530 and the wire drive portion 550 based on the acquired operation input. When the acquired operation input is an operation input related to the video control device 600, the drive controller 560 transmits the acquired operation input to the main controller 660 of the video control device 600.

The drive controller 560 is a computer capable of executing a program including a processor, a memory, a storage portion capable of storing programs and data, and an input/output control portion. The function of the drive controller 560 is realized by the processor executing the program. At least some functions of the drive controller 560 may be realized by a dedicated logic circuit.

The drive controller 560 may further have a configuration other than the processor, the memory, the storage portion, and the input/output control portion. For example, the drive controller 560 may further have an image calculation portion that performs a part or all of image processing and image recognition processing. By further having an image calculation portion, the drive controller 560 can execute specific image processing and image recognition processing at high speed. The image calculation portion may be mounted on a separate hardware device connected by a communication line.

### [Video Control Device 600]

FIG. 11 is a functional block diagram of the video control device 600.

The video control device 600 includes a third adapter 610, an imaging processing portion 620, a light source portion 630, and a main controller 660.

The third adapter 610 is an adapter to which a connecting portion 671 provided on the proximal end side of the video control cable 670 is detachably connected. The video control cable 670 includes a video cable 680 and a light cable 690.

The imaging processing portion 620 is connected to the video cable 680. The imaging processing portion 620 converts the imaging signal acquired from the imaging portion 111c of the distal end portion 111 into a captured image via the imaging cable 173 and the video cable 680.

The light source portion 630 is connected to the light cable 690. The light source portion 630 generates illumination light to be applied to the imaging target. The illumination light generated by the light source portion 630 is guided to the illumination portion 111b of the distal end portion 111 via the light cable 690 and the light guide 174.

The main controller 660 is a computer capable of executing a program equipped with a processor, memory, and the like. The function of the main controller 660 is realized by the processor executing the program. At least some functions of the main controller 660 may be realized by a dedicated logic circuit.

The main controller 660 can perform image processing on the captured image acquired by the imaging processing portion 620. The main controller 660 can generate a GUI image or a CG image for the purpose of providing information to the surgeon S. The main controller 660 can display the captured image, GUI image, and CG image on the display device 900.

The main controller 660 is not limited to an integrated hardware device. For example, the main controller 660 may be configured by partially separating it as a separate hardware device and then connecting the separate hardware device via a communication line. For example, the main controller 660 may be a cloud system in which separate storage portions 563 are connected by a communication line.

The main controller 660 may further have a configuration other than the processor and the memory. For example, the main controller 660 may further have an image calculation portion that performs a part or all of the image processing and the image recognition processing performed by the processor. By further having an image calculation portion, the main controller 660 can execute specific image processing and image recognition processing at high speed. The image calculation portion may be mounted on a separate hardware device connected by a communication line.

### [Operation of Endoscope System 1000]

Next, the operation of the endoscope system 1000 of this embodiment will be described. Specifically, a procedure for observing and treating an affected area formed on a tube wall in the large intestine using the endoscope system 1000 will be described.

The surgeon S inserts the insertion portion 110 of the endoscope 100 into the large intestine from the anus of the patient P from the distal end. As shown in FIG. 2, the surgeon S moves the insertion portion 110 while observing the captured image displayed on the display device 900 and operating the internal soft portion 119 with the right hand R to bring the distal end portion 111 closer to the affected area. Further, the surgeon S operates the operation device 300 by the left hand L to bend the bending portion 112 as necessary.

The stand 200 and the connector 240 can be located away from the trolley 800 and near the patient P and the surgeon S. Therefore, the surgeon S can easily change the position and posture of the internal soft portion 119 by rotating the connector 240 in the horizontal direction by operating the internal soft portion 119 of the endoscope 100 with the right hand R Since the connector 240 is supported at a position higher than the operation table T, the first connector 260 can be brought closer to the anus of the patient P as shown in FIG. 2. By bringing the first connector 260 close to the anus of the patient P, the insertion portion 110 of the endoscope 100 can be effectively utilized up to the proximal end side, so that the total length of the insertion portion 110 of the endoscope 100 can be shortened.

In the endoscope system 1000, since the endoscope 100 and the operation device 300 are separated, the surgeon S can operate the endoscope 100 and the operation device 300 independently without being affected by each other. Therefore, the operation device 300 can assign the twisting motion of the wrist of the left hand L of the surgeon S, which has been conventionally used for different endoscope operations, to the operation of bending the bending portion 112 in the LR direction. The twisting motion of the wrist makes it easy for the surgeon S to apply a force to rotate the operation portion main body 320, and the burden is small.

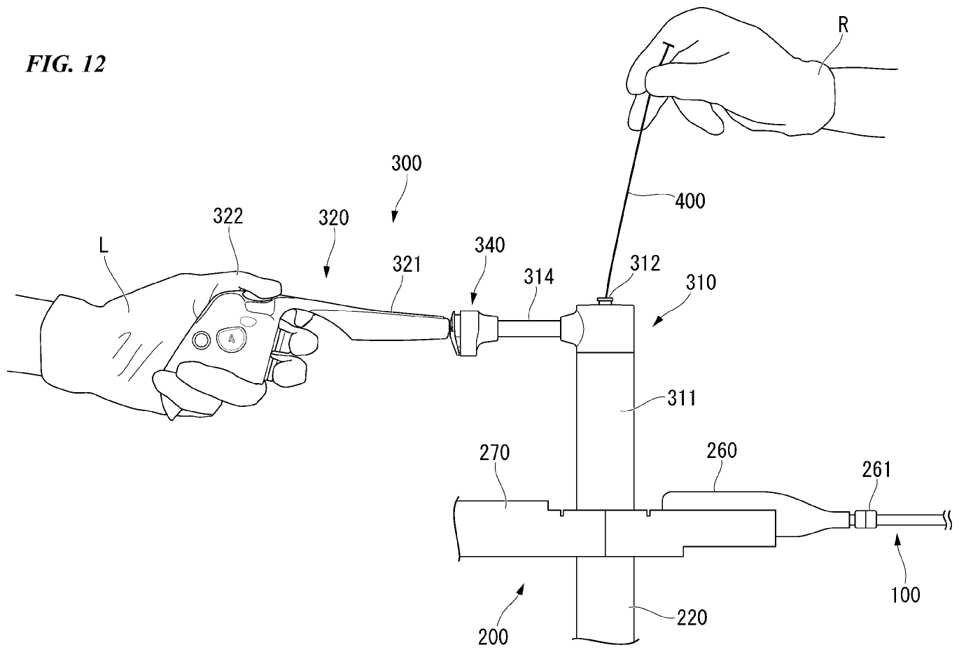

FIG. 12 is a diagram showing the operation device 300 and the treatment tool 400 that are operated at the same time.

The forceps opening 312 into which the treatment tool 400 is inserted is provided in the operation device 300. Therefore, the surgeon S can operate the treatment tool 400 with the right hand R while operating the operation device 30) with the left hand L.

The operation portion main body 320 is supported by the support portion 310, and remains in the current position even when the surgeon S releases the left hand L from the operation portion main body 320. Therefore, the surgeon S is freed from the burden of having to hold the operation device 300 at all times.

According to the endoscope system 1000 of the present embodiment, the operation device 300 to which the operation of the surgeon S is input can be easily handled and intuitively operated.

Although the first embodiment of the present invention has been described in detail with reference to the drawings, the specific configuration is not limited to this embodiment and includes design changes and the like within a range not deviating from the gist of the present invention. In addition, the components shown in the above-described embodiments and modifications can be appropriately combined and configured.

Although the procedure in which the treatment target is the large intestine is described in detail in the above embodiment, the treatment target of the procedure using the endoscope system is not limited to this. The procedure using the endoscopic system can target any body cavity including the upper gastrointestinal tract, the lower gastrointestinal tract, and the like.

What is claimed is:

1. An endoscope system comprising:
a drive cable;
an endoscope that has a bending portion that is capable of bending and is driven by the connected drive cable;
a drive device that electrically drives the connected drive cable;
a trolley equipped with the drive device;
a stand that connects the drive cable and the endoscope and is capable of being installed at a position away from the trolley; and
an operation device that is communicably connected to the drive device and attached to the stand.

2. The endoscope system according to claim 1, wherein the operation device includes:
a grip;
a shaft provided in front of the grip; and
an operation input portion to which an operation of bending the bending portion by rotating the shaft along a longitudinal axis is input.

3. The endoscope system according to claim 2, wherein the drive device controls an amount of bending that bends the bending portion based on an amount of rotation of a roll rotation that has been input.

4. The endoscope system according to claim 2, wherein the drive device bends the bending portion in a left-right direction based on a direction of a roll rotation that has been input.

5. The endoscope system according to claim 2, wherein the operation device has an operation lever to which an operation of bending the bending portion in a vertical direction is input on an upper surface of the grip.

6. The endoscope system according to claim 2, wherein
the operation device has an air supply/water supply button on a front surface of the front of the grip, and
the drive device controls air supply/water supply of the endoscope based on an operation input from the air supply/water supply button.

7. The endoscope system according to claim 2, wherein
the operation device has a suction button on a front surface of the front of the grip, and
the drive device controls suction of the endoscope based on an operation input from the suction button.

8. The endoscope system according to claim 1, wherein the operation device includes a forceps opening into which a treatment tool can be inserted into a lumen of the endoscope.

9. The endoscope system according to claim 2, wherein the operation device includes:
a support column connected to the stand; and
an arm that connects the support column and the operation input portion, and has a rigidity smaller than those of the support column and the shaft.

10. A controller that controls an endoscope including a bending portion that is capable of bending, the controller including:
a support portion;
a grip;
a shaft provided in front of the grip; and
an operation input portion which is supported by the support portion and to which an operation of bending the bending portion by rotating the shaft along a longitudinal axis is input.

11. The controller according to claim 10, wherein the support portion includes a forceps opening into which a treatment tool can be inserted into the lumen of the endoscope.

12. The controller according to claim 10, wherein
the support portion is formed in a columnar shape, and
the shaft is connected to the operation input portion so that the longitudinal axis of the shaft intersects a central axis of the support portion.

13. The controller according to claim 10, wherein the grip and the shaft are connected so that a front surface of the grip is below the shaft.

14. The controller according to claim 12, further comprising an operation lever on an upper surface of the grip to which an operation of bending the bending portion in a vertical direction is input.

15. The controller according to claim 10, further comprising an air supply/water supply button to which an operation of supplying air/water to the endoscope is input at a front surface of the front of the grip.

16. The controller according to claim 10, further comprising a suction button on a front surface of the front of the grip to which an operation of sucking the endoscope is input.

17. A stand that supports a controller that controls an endoscope including a bending portion that is capable of bending, the stand comprising:
a controller including a grip, a shaft provided in a front of the grip, and an operation input portion to which an operation of bending the bending portion by rolling the shaft along a longitudinal axis is input; and
a pole that supports the controller.

18. The stand according to claim 17, further comprising a support column that is connected to the pole,
wherein the support column is provided with a forceps opening into which a treatment tool can be inserted into a lumen of the endoscope.

19. The stand according to claim 18, wherein
the support column is formed in a columnar shape, and
the shaft is connected to the operation input portion so that the longitudinal axis of the shaft intersects a central axis of the support column.

20. The stand according to claim 17, further comprising an operation lever on an upper surface of the grip to which an operation of bending the bending portion in a vertical direction is input.
